SEQUENCE LISTING (15 sequences)

SEQ ID NO: 1 - DNA, 513 bp, Eimeria tenella
atgggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggaattaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaagagcagaacaaagcagatgcgctgacaacggcacttgccttcgctgagtacctgtaccagggcggcttctaa

SEQ ID NO: 2 - protein, 170 aa, Eimeria tenella
MGEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGGFLIKTPNDNVVIALYDEEKEQNKADALTTALAFAEYLYQGGF

SEQ ID NO: 3 - DNA, 1064 bp, Artificial Sequence
  Chemically Synthesized
gacagcaaatgggtcgggcacgagtcttcattgtttgtagtttctttgtatttccttactcagttaaaatgggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggaattaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaagagcagaacaaagcagatgcgctgacaacggcacttgccttcgctgagtacctgtaccagggcggcttctaattgatctccagtgcacaaccacttgatgagaaggaaaaacctttcataacaacaacttcccccagtgttgccacacagggagaagagagacgcacaacttctctacaaatagcggacagcgtattgcacaccctgacctttgtttattgaagagggtgtagggggaggagcatcagcaggcagcagctttgggcggtctggacagttcgccatggagggagagctgtgtagacactcgagagcagcagcagcagcacggttaagtggcagacgcagagacgcctttgttgtacaacttctctctcacccgcgtttgttgtagagaggagtatttattatgaatgcatatccagcaaacaacgaggcaaacagcgggtgcttactgccgtgcaaatgatacgcacaccaccaaccatttaataagtgcttttcttaatatggcttgacgctcccagcgaaaaaaaaaacggctgctaacaaagc

SEQ ID NO: 4 - DNA, 513 bp, Artificial Sequence; identical to SEQ ID NO: 1
  Chemically Synthesized

SEQ ID NO: 5 - DNA, 68 bp, Artificial Sequence
  Chemically Synthesized
gacagcaaatgggtcgggcacgagtcttcattgtttgtagtttctttgtatttccttactcagttaaa

SEQ ID NO: 6 - DNA, 483 bp, Artificial Sequence
  Chemically Synthesized
ttgatctccagtgcacaaccacttgatgagaaggaaaaacctttcataacaacaacttcccccagtgttgccacacagggagaagagagacgcacaacttctctacaaatagcggacagcgtattgcacaccctgacctttgtttattgaagagggtgtagggggaggagcatcagcaggcagcagctttgggcggtctggacagttcgccatggagggagagctgtgtagacactcgagagcagcagcagcagcacggttaagtggcagacgcagagacgcctttgttgtacaacttctctctcacccgcgtttgttgtagagaggagtatttattatgaatgcatatccagcaaacaacgaggcaaacagcgggtgcttactgccgtgcaaatgatacgcacaccaccaaccatttaataagtgcttttcttaatatggcttgacgctcccagcgaaaaaaaaaacggctgctaacaaagc

SEQ ID NO: 7 - protein, 192 aa, Artificial Sequence
  Chemically Synthesized
QQMGRARVFIVCSFFVFPYSVKMGEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGGFLIKTPNDNVVIALYDEEKEQNKADALTTALAFAEYLYQGGF

SEQ ID NO: 8 - DNA, 576 bp, Artificial Sequence
  Chemically Synthesized
atgaaaaaatatttattgggaataggtctaatattagccttaatagcatgtaagcaaaatgttagcggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggaattaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaagagcagaacaaagcagatgcgctgacaacggcacttgccttcgctgagtacctgtaccagggcggcttctaa

SEQ ID NO: 9 - DNA, 66 bp, Artificial Sequence
  Chemically Synthesized
atgaaaaaatatttattgggaataggtctaatattagccttaatagcatgtaagcaaaatgttagc

SEQ ID NO: 10 - DNA, 510 bp, Artificial Sequence
  Chemically Sythesized
ggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggaattaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaagagcagaacaaagcagatgcgctgacaacggcacttgccttcgctgagtacctgtaccagggcggcttctaa

SEQ ID NO: 11 - protein, 191 aa, Artificial Sequence
  Chemically Synthesized
MKKYLLGIGLILALIACKQNVSGEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGGFLIKTPNDNVVIALYDEEKEQNKADALTTALAFAEYLYQGGF

SEQ ID NO: 12 - protein, 22 aa, Artificial Sequence
  Chemically Synthesized
MKKYLLGIGLILALIACKQNVS

SEQ ID NO: 13 - protein, 169 aa, Artificial Sequence
  Chemically Synthesized
GEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGGFLIKTPNDNVVIALYDEEKEQNKADALTTALAFAEYLYQGGF

SEQ ID NO: 14 - DNA, 34 bp, Artificial Sequence
  Chemically Synthesized
gctttgttagcagccgttagaagccgccctggta

SEQ ID NO: 15 - DNA, 36 bp, Artificial Sequence
  Chemically Synthesized
gacagcaaatgggtcgatgggtgaagaggctgatac

(19) United States
(12) Patent Application Publication
(10) Pub. No.: US 2018/0153945 A1
(43) Pub. Date: Jun. 7, 2018

(54) ORAL E. COLI VECTOR-BASED VACCINE FOR PREVENTION OF COCCIDIOSIS IN POULTRY

(71) Applicants: The United States of America, as represented by the Secretary of Agriculture, Washington, DC (US); US Biologic, Memphis, TN (US)

(72) Inventors: HYUN S. LILLEHOJ, WEST FRIENDSHIP, MD (US); WOOHYUN KIM, ELLICOTT CITY, MD (US); CHRIS PRZYBYSZEWSKI, MEMPHIS, TN (US); D.STEVEN ZATECHKA, JR., CORDOVA, TN (US)

(21) Appl. No.: 15/828,778

(22) Filed: Dec. 1, 2017

Related U.S. Application Data
(60) Provisional application No. 62/429,941, filed on Dec. 5, 2016.

(51) Int. Cl. A61K 35/741; A61K 39/108; A61K 39/012

(52) U.S. Cl. CPC A61K 35/741; A61K 2039/552; A61K 39/012; A61K 39/0258

(57) ABSTRACT

The invention relates to recombinant vaccines capable of presenting all, or antigenic portions of, the Eimeria tenella 3-1e, or profilin. Also provided are methodologies of using the vaccines for oral administration to poultry and other targets in the control of coccidiosis. In particular embodiments, recombinant host cells, such as E. coli, expressing all or part of the 3-1e antigen, are provided that can be used as whole-cell vaccines. In some instances, the native 3-1e protein utilized in the vaccines presented herein is molecularly manipulated.

### CROSS-REFERENCE

The present application claims priority to U.S. Provisional Patent Application Ser. No. 62/429,941 filed Dec. 5, 2016, the content of which is expressly incorporated herein by reference.

### BACKGROUND OF THE INVENTION

### Field of Invention

The subject matter disclosed herein provides recombinant vaccines capable of presenting all, or antigenic portions of, the Eimeria tenella 3-1e, or profilin, protein in the development of active immunity to, and control of, coccidiosis. Also provided are methodologies of using the vaccines for oral administration to poultry and other animals in the control of coccidiosis. More particularly, recombinant host cells, such as E. coli, expressing all or part of the 3-1e antigen are provided. In some instances, the 3-1e protein utilized in the vaccines presented herein is molecularly manipulated.

### Background

Poultry coccidiosis is a significant challenge to the United States food supply as a cause of morbidity, mortality and production loss. The disease is the result of infection by one or more of three main species of protozoan parasites in the genus Eimeria—E. tenella, E. maxima, and E. acervulina—and results in systemic and gastrointestinal pathology in growing birds. Restrictions on the use of antimicrobials in food production enacted by health officials and those arising from consumer choices to purchase meat produced without antibiotics has limited the tools veterinarians have today. Few new agents are in development for the future to control this disease. Vaccinations are a possible route, and most current vaccines are modified live strains that provide controlled exposure. However, currently available vaccines, at best, limit disease compared to traditional antibiotic treatment and prophylaxis.

The use of live parasite vaccines and prophylactic medications has historically controlled the spread of disease. Modified live vaccines to coccidiosis often produce subclinical symptomology in the birds, including enteric ulcers, which slows feed conversion. The use of antibiotic medications has been slowed by continued market and regulatory pressures and the consequent reduced susceptibility of the pathogens to these agents. Further, the development of parasite strains resistant to drug treatments, and immune-evasive mutations introduced in response to live parasite treatments, can limit the effectiveness of such options. Consequently, the poultry market has placed enormous pressure on the poultry industry to find effective prophylactic treatments, especially as corporate poultry consumers and producers are moving away from poultry treated by some widely-used prophylactic medications.

Discontinuing use of prophylactic medications can lead to poultry disease prevention gaps—the time between application of a vaccine and development of an effective immune response—across the U.S. Further, as the human population continues to rise coupled with an increase in chickens as a source for dietary protein, the cost of controlling coccidiosis (production and delivery) will also rise, thus increasing the need for cost effective treatment and prevention strategies (Shirley and Lillehoj, Avian Path. (2012) 41:111-21; Wallach, Trends Parasitol. (2010) 26:382-7).

Subunit vaccines have historically been ineffective in the control of coccidiosis for a variety of reasons, including either the relative lack of potency of the subunit vaccine or—though more effective—the necessary inclusion of potentially expensive and harmful chemical adjuvants. Compared to in ovo vaccine-administered solutions, orally delivered prophylactic vaccine agents are manufactured more cost effectively, offer a significant ease of use, and, if administered with enteric stability, offer targeted elicitation of mucosal immunity. To address these issues, presented herein are compositions and methods for the effective oral administration of a vaccine for the control of coccidiosis in poultry, as caused by Eimeria species.

### SUMMARY OF THE INVENTION

Provided herein are multiple embodiments of the inventions, including a recombinant vaccine comprising a transformed host cell expressing the 3-1e protein (SEQ ID NO: 2), or a protein having at least 95% identity to 3-1e, on its cell surface, wherein 3-1e is encoded by a nucleic acid used to transform the host cell, and a pharmacological carrier. In some embodiments, vaccines provided herein contain an adjuvant. In some embodiments, the host cell is an Escherichia coli cell. In other embodiments, vaccines of the present invention further comprise a probiotic organism of the genus Lactobacillus, for example, L. acidophilus, L. brevis, L. casei, L. crispatus, L. fermentum, L. gasseri, L. plantarum, L. reuteri, L. rhamnzosus, or L. salivarius. In still other embodiments, the vaccine is a killed whole-cell vaccine or a live whole-cell vaccine. In some embodiments, the pharmacological carrier is a hydrocolloid polymer, a plasticizing sugar (such as sucrose or trehalose), or a combination thereof. In a particular embodiment, the pharmacological carrier utilized is sodium alginate. Vaccines of the present invention in which the carrier is a hydrocolloid polymer, the hydrocolloid polymer can be cross-linked using calcium acetate, calcium ascorbate, calcium butyrate, calcium carbonate, calcium chloride, calcium lactate, or calcium sulfate, with cross-linking using calcium butyrate as a particular embodiment.

Another embodiment provided herein is a recombinant vaccine comprising a transformed host cell expressing a 3-1e/OspA hybrid protein (SEQ ID NO: 11), or a protein having at least 95% identity to a 3-1e/OspA hybrid protein, wherein a 3-1e/OspA hybrid protein is encoded by a nucleic acid used to transform the host cell, on its cell surface and a pharmacological carrier. In some embodiments, vaccines provided herein contain an adjuvant. In some embodiments, the host cell is an Escherichia coli cell. In other embodiments, vaccines of the present invention further comprise a probiotic organism of the genus Lactobacillus, for example, L. acidophilus, L. brevis, L. casei, L. crispatus, L. fermentum, L. gasseri, L. plantarum, L. reuteri, L. rhamnzosus, or L. salivarius. In still other embodiments, the vaccine is a killed whole-cell vaccine or a live whole-cell vaccine. In some embodiments, the pharmacological carrier is a hydrocolloid polymer, a plasticizing sugar (such as sucrose or trehalose), or a combination thereof. In a particular embodiment, the pharmacological carrier utilized is sodium alginate. Vaccines of the present invention in which the carrier is a hydrocolloid polymer, the hydrocolloid polymer can be cross-linked using calcium acetate, calcium ascorbate, calcium butyrate, calcium carbonate, calcium chloride, calcium lactate, or calcium sulfate, with cross-linking using calcium butyrate as a particular embodiment.

Also provided herein is an embodiment of producing any of the vaccines described herein. The processes provided include the steps of: culturing a recombinant host cell transformed with DNA encoding 3-1e (SEQ ID NO: 1), DNA encoding a 3-1e/OspA hybrid protein (SEQ ID NO: 8), a DNA sequence encoding a protein having at least 95% identity to 3-1e (SEQ ID NO: 2), or a DNA sequence encoding a protein having at least 95% identity to a 3-1e/OspA hybrid protein (SEQ ID NO: 11); expressing the protein encoded by the recombinant DNA sequence; recovering the host cells produced in the culturing step; and incorporating the host cells expressing the protein in or on a pharmacological carrier. In some embodiments, this method has the further step of incorporating an adjuvant. In some embodiments, the host cell is an Escherichia coli cell. In some embodiments, the pharmacological carrier is a hydrocolloid polymer, a plasticizing sugar (such as sucrose or trehalose), or a combination thereof. In a particular embodiment, the pharmacological carrier utilized is sodium alginate. Vaccines of the present invention in which the carrier is a hydrocolloid polymer, the hydrocolloid polymer can be cross-linked using calcium acetate, calcium ascorbate, calcium butyrate, calcium carbonate, calcium chloride, calcium lactate, or calcium sulfate, with cross-linking using calcium butyrate as a particular embodiment.

Further provided herein is an embodiment which is a method of protecting a recipient against an Eimeria species, comprising: administering any of the recombinant vaccines disclosed herein to a recipient in an amount effective to induce an immune response against the exogenous protein produced by the recombinant vaccine. In particular embodiments, the recipient is a chicken or a turkey. In some embodiments, the further step of administering a probiotic organism of the genus Lactobacillus, such as L. acidophilus, L. brevis, L. casei, L. crispatus, L. fermentum, L. gasseri, L. plantarum, L. reuteri, L. rhamnzosus, or L. salivarius is an additional step of the method. In some embodiments of this method, the recombinant vaccine is administered to the recipient as a live whole-cell formulation at a dose of 5×10³ to 5×10⁹ CFU, or at a dose of 5×10³ to 5×10⁹ cells. In many embodiments, the recombinant vaccine is administered orally.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides a map representing the molecular engineering of the 3-1e coding sequence contig into the pET9c inducible vector. The induction of the T7 RNA polymerase results in the exclusive expression of the 3-1e antigenic protein under the control of the T7 RNA polymerase promoter. Empty-vectored pET9c (EV) was designated as an administration control.

FIG. 2 provides a map representing the molecular engineering of the 3-1e coding sequence contig (CDS) coupled on both the 5′- and 3′-ends to the respective 5′- and 3′-untranslated regions (UTRs). Depending upon the level of expression of the 3-1e protein antigen required to elicit efficacy in the context of an orally-administered vaccine platform, the UTRs may provide a level of stability to the translation of the protein in a recombinant E. coli carrier strain. The induction of the T7 RNA polymerase results in the exclusive expression of the 3-1e antigenic protein (from the CDS) under the control of the T7 RNA polymerase promoter.

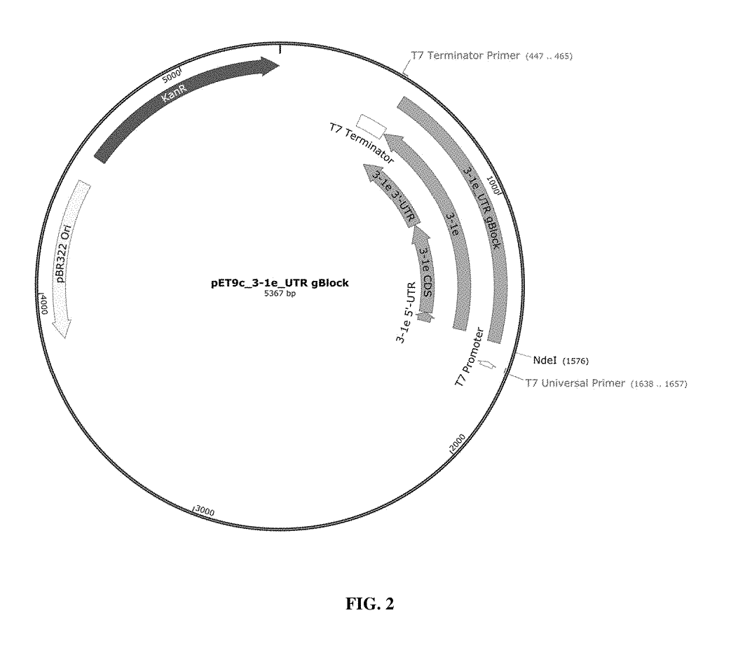

FIG. 3 provides a map representing the molecular engineering of the 3-1e coding sequence contig (CDS) coupled on the 5′-end to the OspA-encoded lipoprotein. The OspA lipoprotein is expressed as a molecular adjuvant in concert with proximal (in-frame) vaccine antigens expressed in fusion. The expression of the resulting fusion construct in a recombinant E. coli carrier strain can thereby enhance the immune reaction in response to the vaccine in the context of an orally-administered vaccine platform.

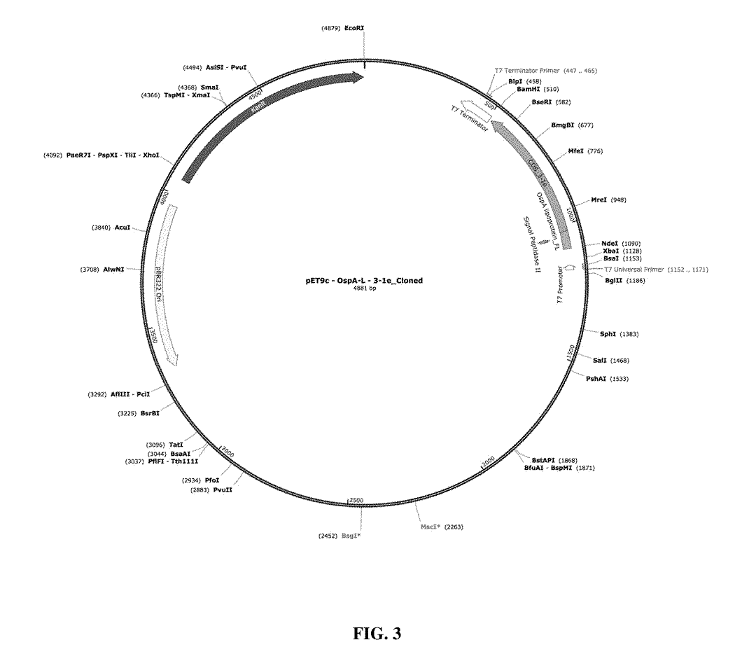

FIG. 4 provides SDS-PAGE and Western blot analyses demonstrating the presence of recombinant 3-1e protein from induced whole-cell lysates. Lane 1: marker. Lane 2: negative control (bacteria transformed with vector pET9c with no insert). Lane 3: exemplary bacteria transformed with vector pET9c containing the 3-1e coding sequence.

FIG. 5 provides a photomicrograph of micro-beads comprising a vaccine of the present invention. The beads present as spherical structures of encapsulated vaccine of physical qualities for hydrocolloidal solutions as carriers for oral vaccine administration. This formulation preparation further imparts a process supporting an anhydrobiotic qualification that is both stable and scalable.

FIGS. 6A, 6B and 6C provide photomicrographs demonstrating delivery of vaccines of the present invention to the digestive tract of chickens. FIG. 6A is a control showing no presence of E. coli or 3-1e protein. FIG. 6B shows tissue collected from a specimen treated with orally delivered 3-1e-expressing E. coli (3-1e stain). FIG. 6C shows tissue collected from a specimen treated with orally delivered 3-1e-expressing E. coli (E. coli stain).

FIG. 7 provides graphs showing immunological responses in chickens. Antibody titers from blood samples in negative control (uninfected), positive control (infected), and empty-vector and 3-1e-expressing-vector E. coli vaccine treated chickens are shown.

FIG. 8 provides graphs showing reduction in the lesion scores in 3-1e vaccinates relative to the controls.

FIG. 9 provides graphs showing reduction in Eimeria oocyst shedding in 3-1e vaccinates relative to the controls.

FIG. 10 provides graphs showing weight gain in 3-1e vaccinates relative to the controls.

### DETAILED DESCRIPTION OF THE INVENTION

Provided herein are recombinant vaccines capable of presenting all, or antigenic portions of, the Eimeria tenella 3-1e, or profilin, protein in the development of active immunity to, and control of, coccidiosis. More particularly, recombinant host cells, such as E. coli, expressing all or part of the 3-1e antigen are provided. In some instances, the 3-1e protein utilized in the vaccines presented herein is molecularly manipulated. In some instances, vaccines of the present invention, comprise other components, such as stabilizers and adjuvants. Also provided are methodologies of using the vaccines for oral administration to poultry and other animals in the control of coccidiosis.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This invention teaches methods and describes tools for producing genetically altered strains of A. pullulans.

As used in the specification and claims, use of the singular “a”, “an”, and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The term “about” is defined as plus or minus ten percent of a recited value. For example, about 1.0 g means 0.9 g to 1.1 g and all values within that range, whether specifically stated or not.

The term “a nucleic acid consisting essentially of”, and grammatical variations thereof, means nucleic acids that differ from a reference nucleic acid sequence by 20 or fewer nucleic acid residues and also perform the function of the reference nucleic acid sequence. Such variants include sequences which are shorter or longer than the reference nucleic acid sequence, have different residues at particular positions, or a combination thereof.

The terms “3-1e” and “profilin” are synonyms and refer to the protein defined herein as SEQ ID NO: 2 and encoded by the DNA of SEQ ID NO: 1 (or any version of SEQ ID NO: 1 with base substitutions that result in a protein with a sequence identical to SEQ ID NO: 2). These terms also refer to modified versions of these SEQ ID NOs, such as those comprising regulatory nucleic acids or proteins (and the nucleic acids encoding them) containing additional moieties allowing for cell-surface presentation or immunogenicity-enhancement. In such situations, the additional component is indicated by a relevant signifier (e.g., 3-1e/OspA). Specific examples of such modified sequences are provided as SEQ ID NO: 3 (3-1e with 5′ and 3′ untranslated regions), SEQ ID NO: 8 (3-1e/OspA encoding nucleic acid) and SEQ ID NO: 11 (3-1e/OspA hybrid protein).

As used herein, the term “probiotic” is defined as one or more beneficial bacterium/bacteria and/or isolates of the same that provide a therapeutic benefit to the recipient. Probiotics as used herein can also comprise media, carriers, or other vehicles suitable for use in the intended recipient.

As used herein, the term “poultry” refers to one bird, or a group of birds, of any type of domesticated birds typically kept for egg and/or meat production. For example, poultry includes chickens, ducks, turkeys, geese, bantams, quail, pheasant, pigeons, or the like, preferably commercially important poultry such as chickens, ducks, geese and turkeys.

As used herein, the term “livestock” can include any commercially important animal such as poultry, swine or cattle.

The terms “isolated”, “purified”, or “biologically pure” as used herein, refer to material that is substantially, or essentially, free from components that normally accompany the referenced material in its native state.

The term “bioactive agent” or “biologically active agent” refers to any substance that is of medical or veterinary therapeutic, prophylactic or diagnostic utility. In some embodiments, a bioactive agent includes a therapeutic agent. As used herein, a therapeutic agent refers to a bioactive agent that, when administered, will cure, or ameliorate, one or more symptoms of a disease or disorder. In some embodiments, a bioactive agent can be a prophylactic agent. As used herein, a prophylactic agent refers to a bioactive agent that, when administered either prevents the occurrence of, or lessens the severity of, a disease or disorder or, if administered subsequent to a therapeutic agent, prevents or retards the recurrence of the disease or disorder. In some instances, a bioactive agent can refer to antigens that elicit an immune response, or proteins that can modulate the immune system, to enhance therapeutic potential. In some embodiments, the administration of the biologically active antigenic agent can elicit an immune response that is either prophylactic to prevent disease contraction and transmission, or therapeutic to resolve existing disease infection.

The term “vaccine” refers to a preparation of immunogenic material capable of stimulating an immune response, administered for the prevention, amelioration, or treatment of disease, such as an infectious disease. The immunogenic material can include, for example, attenuated or killed microorganisms (such as attenuated viruses), or antigenic proteins, peptides or DNA derived from an infectious microorganism. Vaccines can elicit both prophylactic (preventative) and therapeutic responses. Methods of administration vary according to the vaccine, but can include inoculation, ingestion, inhalation or other forms of administration. Inoculations can be delivered by any of a number of routes, including parenteral, such as intravenous, subcutaneous or intramuscular. Vaccines can be administered with an adjuvant to boost the immune response.

For the purpose of this invention, the sequence “identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch, J Mol Biol, (1970) 48:3, 443-53). A computer-assisted sequence alignment can be conveniently performed using a standard software program such as GAP which is part of the Wisconsin Package Version 10.1 (Genetics Computer Group, Madison, Wis., USA) using the default scoring matrix with a gap creation penalty of 50 and a gap extension penalty of 3.

### Molecular Biological Methods

An isolated nucleic acid is a nucleic acid the structure of which is not identical to that of any naturally occurring nucleic acid. The term therefore covers, for example, (a) a DNA which has the sequence of part of a naturally occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transformed or transfected cells, and (iii) cell clones, e.g., as these occur in a DNA library such as a cDNA or genomic DNA library.

The term recombinant nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

In practicing some embodiments of the invention disclosed herein, it can be useful to modify the genomic DNA of a recombinant strain of the host cell producing the immunogenic protein of the vaccine (e.g., 3-1e protein). In preferred embodiments, such a host cell is E. coli. Such modification can involve deletion of all or a portion of a target gene, including but not limited to the open reading frame of a target locus, transcriptional regulators such as promoters of a target locus, and any other regulatory nucleic acid sequences positioned 5′ or 3′ from the open reading frame. Such deletional mutations can be achieved using any technique known to those of skill in the art. Mutational, insertional, and deletional variants of the disclosed nucleotide sequences and genes can be readily prepared by methods which are well known to those skilled in the art. It is well within the skill of a person trained in this art to make mutational, insertional, and deletional mutations which are equivalent in function to the specific ones disclosed herein.

Where a recombinant nucleic acid is intended for expression, cloning, or replication of a particular sequence, DNA constructs prepared for introduction into a prokaryotic or eukaryotic host will typically comprise a replication system (i.e. vector) recognized by the host, including the intended DNA fragment encoding a desired polypeptide, and can also include transcription and translational initiation regulatory sequences operably linked to the polypeptide-encoding segment. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient identity to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Hybridization-based screening of genetically altered strains typically utilizes homologous nucleic acid probes with identity to a target nucleic acid to be detected. The extent of identity between a probe and a target nucleic acid can be varied according to the particular application. Identity can be 50%-100%. In some instances, such identity is greater than 80%, greater than 85%, greater than 90%, or greater than 95%. The degree of identity or identity needed for any intended use of the sequence(s) is readily identified by one of skill in the art. As used herein percent sequence identity of two nucleic acids is determined using the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See http://www.ncbi.nih.gov.

Preferred host cells are members of the genus Escherichia, especially E. coli. However, any suitable bacterial or fungal host capable of expressing the described proteins can be utilized. Even more preferably, non-pathogenic and non-toxigenic strains of such host cells are utilized in practicing embodiments of the disclosed inventions. Examples of workable combinations of cell lines and expression vectors are described in Sambrook et al. (1989); Ausubel et al. (Eds.) (1995) Current Protocols in Molecular Biology, Greene Publishing and Wiley Interscience, New York; and Metzger et al. (1988) Nature, 334: 31-36. Recombinant host cells, in the present context, are those which have been genetically modified to contain an isolated nucleic molecule of the instant invention. The nucleic acid can be introduced by any means known to the art which is appropriate for the particular type of cell, including without limitation, transformation, lipofection, electroporation or any other methodology known by those skilled in the art.

Recombinant Vaccines

Provided herein are recombinant vaccines and methodologies for their use. In certain embodiments the recombinant vaccines are bacterial cells, such as E. coli and Bacillus subtilis, transformed with a vector capable of expressing a 3-1e (“profilin”) antigen on their surface. Some vectors useful in the present invention can be integrated into the genome by, for example, insertion of exogenous DNA comprising an open reading frame encoding the 3-1e protein or a portion thereof. Other vectors useful in practicing the inventions disclosed herein can be non-integrating nucleic acids, for example self-replicating plasmids, containing exogenous DNA comprising an open reading frame encoding the 3-1e protein or a portion thereof. In preferred embodiments, the exogenous DNA also contains a sequence of DNA encoding a protein, or portion thereof, operably linked to the 3-1e-encoding DNA that allows for presentation of the 3-1e protein on the surface of the recombinant bacterial cell, such as a cell-wall anchoring protein, cell membrane anchoring protein, or cell wall sorting signal. The 3-1e-expressing and presenting recombinant bacterial cells can then be utilized as an oral vaccine for subjects in need of vaccination, particularly poultry such as chickens and turkeys.

In some embodiments, the vaccines of the present invention can be applied to a subject as a whole-cell bacteria expressing a 3-1e protein (SEQ ID NO: 2, SEQ ID NO: 11, SEQ ID NO: 13, etc.), preferably as a cell-surface antigen. “Whole-cell bacteria” refers to bacterial cells that retain all or much of their cellular integrity and are capable of presenting the recombinant protein of the vaccine (e.g., 3-1e). Whole-cell bacterial versions of the vaccines of the present invention include both live whole-cell bacteria and killed whole-cell bacteria.

The immunogenically effective amounts of vaccines disclosed herein can vary based upon multiple parameters. In general, however, effective amounts per dosage unit can be about 10² to 10¹⁴ colony forming units (cfu), about 5.0×10² to 5.0×10¹⁰ cfu, about 1.0×10⁶ cfu to 1.0×10⁹ cfu, and about 5.0×10⁶ cfu to 1.0×10⁹ cfu. These amounts can refer to the same number of killed cells. One, two, or more dosage units can be utilized in practicing the methodologies of the present invention. If two dosage units are selected, then vaccination at about day 1 post-hatch and again at about one week to two weeks of age is preferred. A dosage unit can readily be modified to fit a desired volume or mass by one of skill in the art. Regardless of the dosage unit parameters, vaccine compositions disclosed herein can be administered in an amount effective to produce an immune response to the presented antigen (e.g., 3-1e protein). An “immunogenically effective amount” or “effective amount” of a vaccine as used herein, is an amount of a vaccine that provides sufficient levels of antigenic protein to produce a desired result, such as induction of, or increase in, production of antibody specific to the antigen, protection against coccidiosis, as evidenced by a reduction in gastrointestinal lesions, increased weight gain, and decreased oocyst shedding and other indicators of reduction in pathogenesis. Amounts of vaccine capable of inducing such effects are referred to as an effective amount, or immunogenically effective amount, of the vaccine.

Dosage levels of active ingredients (e.g., the bacterium or the amount of antigen) in vaccine compositions disclosed herein, can be varied by one of skill in the art to achieve a desired result in a subject or per application. As such, a selected dosage level can depend upon a variety of factors including, but not limited to, formulation, combination with other treatments, severity of a pre-existing condition, and the presence or absence of adjuvants. In preferred embodiments, a minimal dose of vaccine is administered. As used herein, the term “minimal dose” or “minimal effective dose” refers to a dose that demonstrates the absence of, or minimal presence of, toxicity to the recipient, but still results in producing a desired result (e.g., protective immunity). Minimal effective doses, or minimum immunizing doses, of the recombinant vaccines provided herein can include doses of, in colony forming units (CFU), 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², 1×10¹³, 5×10¹³, or more. The minimal effective doses can also be any individual CFU within the range of 1×10²-5×10¹³. Determination of a minimal dose is well within the capabilities of one skilled in the art.

Vaccine Formulations

In some instances, vaccines of the present invention also contain or comprise one or more adjuvants, which includes any material included in the vaccine formulation that enhances an immune response in the recipient that is induced by the vaccine. In some instances, such adjuvants can include proteins other components expressed by the vaccine host cell. Non-limiting examples of such adjuvants can include engineered proteins in which the 3-1e protein is expressed as a fusion protein operably linked with immunity-enhancing moieties such as the amino-terminal twenty-two (22) amino acids of the OspA protein (SEQ ID NO: 12 (OspA); SEQ ID NO: 11 (3-1e/OspA fusion protein)). In other embodiments, the host cell can comprise additional molecularly engineered proteins. Other adjuvants can be included as an extra component of the vaccine, whether added to a formulation or expressed by a host cell. Such adjuvants can include, for example, AB5 toxins (e.g., cholera toxin), E. coli heat labile toxin, monophosphoryl lipid A, flagellin, c-di-GMP, inflammatory cytokines, chemokines, definsins, chitosan, carbopol (e.g., CARBIGEN) and combinations of these. Any relevant adjuvant known in the art can be utilized in practicing the inventions disclosed herein.

Vaccine compositions of the present invention can also comprise substrates or carriers in addition to the recombinant vaccine. In some instances a vaccine is coated or layered on the substrate or carrier. As used herein, the term “substrate” refers to a solid or semi-solid support composition, such as a carrier, onto which a vaccine can be applied. Non-limiting examples of substrates include generally-termed forms such as pellets, tablets, kibbles, chewables, powders and beads, as well as specific materials such as microcrystalline cellulose (MCC), plant-based products and soil-based products (e.g., clays). Preferably, substrates or carriers are non-toxic to the recipient. Thus, in some embodiments, vaccines of the present invention are delivered to a target (e.g., poultry) via oral administration of a substrate coated with a 3-1e protein-presenting recombinant vaccine. In some instances the vaccine compositions including substrates can be presented to a target for ingestion via suspension in drinking water.

Vaccine compositions provided herein can also include components that stabilize the vaccine formulation, providing stability to the 3-1e antigen, the recombinant host cell expressing the antigen, or both. Stabilizers can, in some embodiments, also be carriers. In some embodiments, a plasticizing sugar, such as sucrose or trehalose is utilized. In other embodiments, a hydrocolloid or hydrocolloid polymer is used as a stabilizer. Non-limiting examples of natural and synthetic hydrocolloids include agar, carrageenan, chitosan, gelatin, gums, polyvinyl pyrrolidones, starches, polysaccharides, such as alginic acid, sodium alginate and calcium alginate, cellulose and cellulose derivatives, such as ethyl cellulose, methyl cellulose, hydroxypropylmethyl cellulose (HPMC), hydroxy-propyl cellulose (HPC), and carboxymethylcellulose (CMC); polyethylene glycol (PEG), and mixtures thereof. Any suitable plasticizing sugar, hydrocolloid, or combinations thereof can be utilized in practicing embodiments of the invention where such stabilizers are part of the recombinant vaccine composition.

In some instances, hydrocolloids and hydrocolloid polymers are cross-linked to facilitate stabilization, encapsulation, or other structural features of the vaccine composition. Such cross-linking can, for example, be performed using a divalent cation such as calcium to structurally link the polymeric bonds of a hydrocolloid polymer. In a specific embodiment of the present invention a vaccine composition comprising sodium alginate cross-linked with a calcium salt is utilized. Exemplary, but non-limiting, calcium salts include calcium acetate, calcium ascorbate, calcium butyrate, calcium carbonate, calcium chloride, calcium lactate, and calcium sulfate.

Thus, in some embodiments of the present invention, vaccine compositions containing a 3-1e protein-presenting recombinant host cell can also include one or more of a substrate/carrier, a stabilizer/carrier, and an adjuvant. Exemplary vaccines formulations can be found in PCT publication WO 2015/200770, herein specifically incorporated by reference.

Probiotics

The vaccine compositions and methodologies provided herein can also include one or more probiotic bacteria from one or more species. Bacteria useful in such embodiments can be selected based on their ameliorative or preventative capabilities in addressing adverse effects of vaccine treatment including, but not limited to, gastrointestinal (GI) tract lesion development, GI inflammation, secondary infections, decreased body weight gain or feed efficiency in poultry, morbidity, or mortality.

In preferred embodiments, probiotic bacteria utilized are lactic acid bacteria, generally including Gram positive, acid-tolerant bacteria. In particular, members of the genus Lactobacillus are the probiotic bacteria. Exemplary, but non-limiting, species include L. acidophilus, L. brevis, L. casei, L. crispatus, L. fermentum, L. gasseri, L. plantarum, L. reuteri, L. rhamnzosus, and L. salivarius. Lactic acid bacteria for use in the present invention can be commercially available or obtained and isolated from the environment (e.g., poultry GI normal flora).

Probiotics can be co-administered with vaccine compositions of the present invention, either in separate formulations or a single formulation. When a probiotic and a vaccine are co-administered in separate formulations, they can be administered simultaneously, or within seconds, minutes or hours of each other. Alternately, probiotics can be independently administered from vaccine compositions, for example in separate administrations separated by days or weeks. Probiotics can be administered in multiple doses at different times, for example prior to vaccination and post-vaccination, prior to vaccination and at the same time as vaccination, or at the same time as vaccination and post-vaccination. Administration of multiple separate probiotic formulations can be separated for anywhere from two to thirty days.

Vaccination Methodologies

The present disclosure provides compositions for vaccinating targets (e.g., poultry) with a recombinant vaccine presenting the E. tenella protein, 3-1e, or antigenic fragments thereof. Thus, the compositions provided herein can be utilized to induce immunity to E. tenella, and more generally, the disease coccidiosis in targets to which the antigen is provided. In preferred embodiments, vaccines of the present invention, are provided for oral ingestion, such as through drinking water. Application of a vaccine to a subject can result in the development of immunity to the 3-1e protein, preferably development of a mucosal immune response. Application of the vaccines of the present invention can be provided at multiple times or in a single dosage. Application of the vaccines provided to poultry herein can occur for the first time about day 1 post-hatch or any time thereafter. Application can be performed before, during or after the development of Eimeria-caused coccidiosis.

The following examples are offered to illustrate, but not to limit the invention.

### EXAMPLES

### Example 1: Molecular Constructs and Engineering

Construction and engineering of the plasmid constructs employed the use of the New England BioLabs NEBuilder® HiFi DNA Assembly Cloning Kit and the use of the NEBuilder® Primer Design interactive tool using gBlock contigs from Integrated DNA Technoloiges (IDT).

The plasmid construct, pET-32a(+)/3-1e served as the template from which the pET9c/3-1e was re-engineered. Briefly, the full 3-1e (also known as “profilin”) coding sequence from Eimeria tenella (SEQ ID NO: 1) including the 5′ and 3′ UTR regions (SEQ ID NO: 3) was originally deposited (May 2001) into GenBank under Accession AF113613 (www.ncbi.nlm.nih.gov/nuccore/5081395) and used in the construction of pET-32a(+)/3-1e. The updated (November 2013) Accession KF493900 (www.ncbi.nlm.nih.gov/nuccore/kf493900) was referenced and accessed to identify the complete coding sequence representing the 3-1e mRNA, in alignment with the original AF113613 contig. The consensus sequence was submitted to the New England Biolabs NEBuilder® online Assembly Tool portal (nebuilder.neb.com/) for the generation of predicted 5′ and 3′ (3-1e-to-pET9c expression plasmid) overlapping regions to be employed in the cloning of the 3-1e CDS into the pET9c inducible expression system; overlapping sequences generated were: FWD gctttgttagcagccgTTAGAAGCCGCCCTGGTA (SEQ ID NO: 14), and REV gacagcaaatgggtcgATGGGTGAAGAGGCTGATAC (SEQ ID NO: 15), where capital letters represent the 3-1e gene-specific primer. Primers were then used in the bioinformatics generation of a predicted synthetic 3-1e gBlock® gene fragment constructed by IDT (Integrated DNA Technologies, Coralville, Iowa). Successful cloning of the synthetic 3-1e gBlock® gene fragment into the NdeI-linearized pET9c utilized the NEBuilder® HiFi DNA Assembly Cloning Kit (www.neb.com/products/e5520-nebuilder-hifi-dna-assembly-cloning-kit#pd-interactive-tools, New England BioLabs, Ipswich, Mass.), per manufacturers instruction.

### Example 2: 3-1e Molecular Expression System

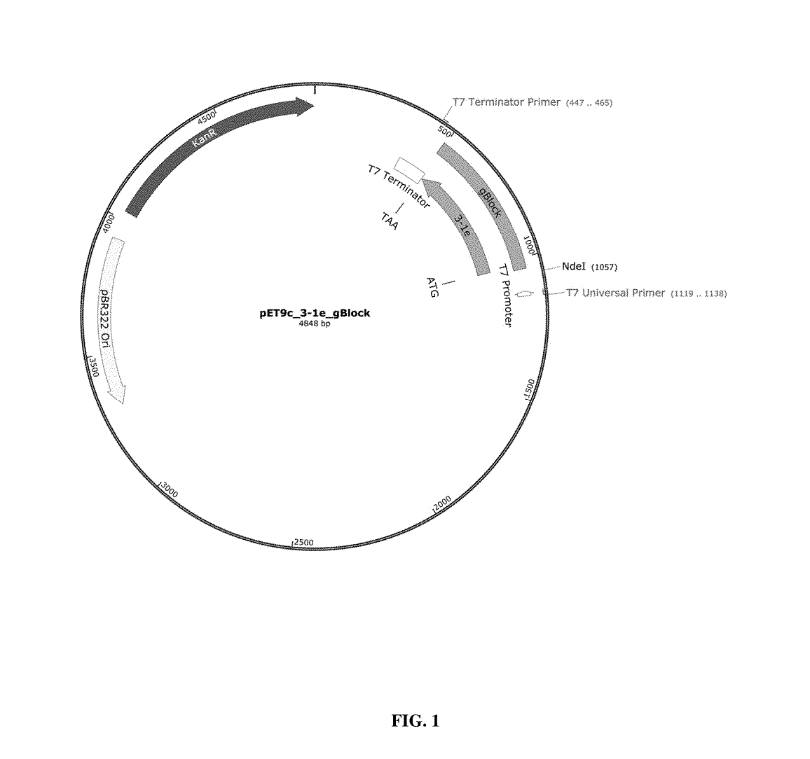

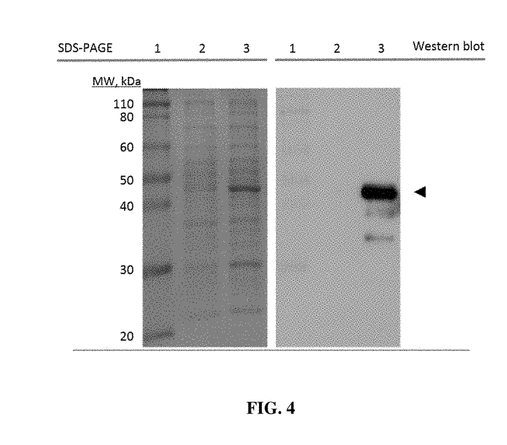

The engineered pET9c/3-1e expression plasmid construct (FIG. 1) was used to transform the BL21(DE3)pLysS strain of competent E. coli (Life Technologies/Thermo Fisher Scientific, Carlsbad, Calif.). Colonies (clones) were isolated and scaled under non-inducing conditions to generate plasmid mini-preps from which transformants were validated for accuracy by sequencing the 3-1e insertion contig using primers against the flanking T7 Promoter and T7 Terminator regions. Such strains present SEQ ID NO: 2 as an antigen. Passage was scaled as biomass for glycerol stocks, and was cultured under induction conditions, via the T7 expression system (Studier, Protein Expr. Purif. (2005) 41:207-34) for use as the immunogenic bioactive agent in subsequent vaccine production. Upon induction, cultures were harvested, washed free of the culture fluids, and the total protein from the biomass was extracted, denatured under standard procedures, and resolved using SDS-PAGE. The Western blots were probed with an anti-3-1e mAb to reveal a robust level of 3-1e expression (migrating to a kDa of approximately 45) in the vaccine carrier samples (FIG. 4).

### Example 3: Vaccine Formulation

Vaccine cultures were passaged in a non-induction media (TBY)+Kan to below OD600=0.8. Cultures was passaged at a 1:1000 inoculum into production (auto-induction) media (Overnight Express™ Instant TB Medium, EMD-Millipore, Billerica, Mass.)+Kan, reconstituted per manufacturers instruction. Cultures were grown to a density of approximately OD600=15, about 17 hours, during which the T7 promoter induced the enhanced expression of the 3-1e protein as the immunogen.

Induced biomass culture fluids were washed using cold 1×PBS (divalent cation free; 81% Sodium Chloride, 2% Potassium Chloride, 14.5% Sodium Phosphate Dibasic, 2.5% Potassium Phosphate Monobasic, all from Thermo Fisher Scientific, Waltham, Mass.), and pelleted. Concentrated biomass was resuspended in 500 mM Sucrose (Thermo Fisher Scientific)/PBS as anhydrobiotic/osmotic conditioning buffer. Conditioned biomass was either cryo-preserved, or immediately used in the production of vaccine.

For vaccine production and administration, conditioned biomass was formulated in a solution of (in order of addition in deionized water) 500 mM Sucrose, 10% Corn Starch (Thermo Fisher Scientific), and 1.5% Sodium Alginate (Maugel GHB, FMC BioPolymer, Philadelphia, Pa.), and agitated constantly until completely homogenized (about 3 hours at room temperature).

The biomass suspension was then electrosprayed into a volume of 2.0% calcium lactate (Acros Organics, Thermo Fisher Scientific, Pittsburgh, Pa.), or 2.0% calcium butyrate (MP Biomedicals, Santa Ana, Calif.), under the electro-physical parameters of a 10-30 mL/hour flow-rate, 28 kV voltage setting, and a spray distance of approximately 6-7 inches, yielding an enteric matrix.

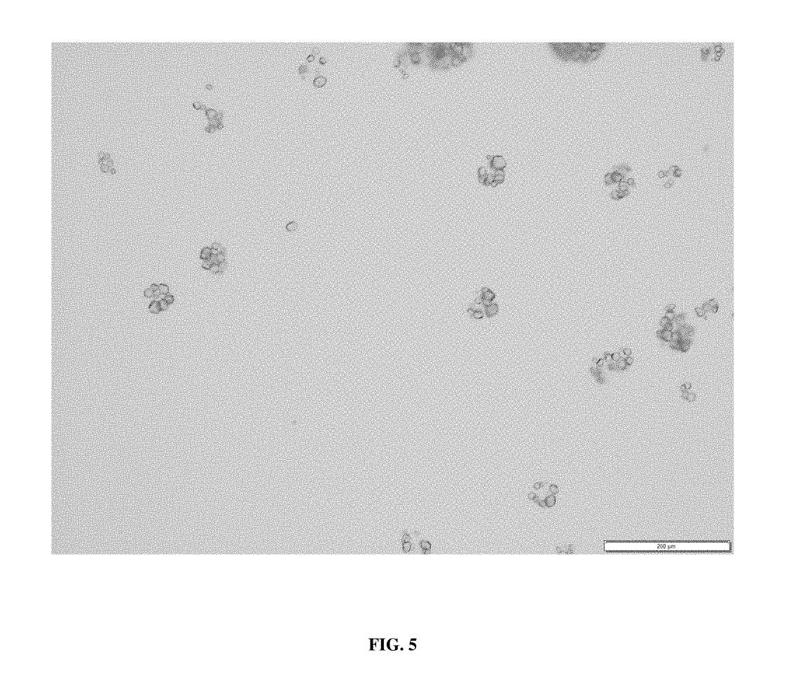

Electrosprayed microbeads were collected, freeze-dried and stored at 4° C. until added to water effectively creating a hydrocolloid suspension of the vaccine for oral administration (controlled experimentally by oral gavage) to poultry. Micro-beads produced by this process are shown in FIG. 5.

Dosage equated to a potency of 1E9 CFU per dose, in a volume of 500 μL, by oral gavage. 3-1e potency via proteomic analysis using Western blotting followed standard procedures as described elsewhere (Lillehoj et al., Avian Dis. (2000) 44:279-89).

Immunohistochemistry (IHC) procedures were conducted on frozen sections of digestive tract as a means to assay targeted oral administration of vaccine, using a polyclonal antibody against 3-1e (ARS-generated anti-sera, Lillehoj, 2000), and secondary staining of goat anti-rabbit Alexa 488.

### Example 3: Animals and Vaccine Testing

1-2 days post-hatch broiler chickens were employed in the efficacy testing of the vaccine. Vaccine administration and testing followed the schedules as presented in the Table 1 below, as part of two independent studies. For schedule 1, three concentrations of the 3-1e vaccine (CFU of 10⁵, 10⁷, and 10⁹, respectively) where tested in groups of eight animals. Treatment groups were inoculated and challenged with E. acervulina (5×10⁴) and compared to control and empty vector (EV) groups. Based on the results of the first phase, the second trial focused on the 10⁷ and 10⁹ CFU variants of the 3-1e vaccine and included 15 birds/group as compared to controls and EV groups. All example data shown reflect the result of a 10⁹ inoculum.

TABLE 1
Vaccination and Experiment Schedule

| Schedule 1 | Schedule 2 |
|---|---|
| Day 0 - Place chicks | Day 0 - Place chicks |
| Day 1 - Vaccination | Day 1 - Vaccination; Weigh |
| Day 2-3 - Intestine collection for | Day 8 - Boosting; Weigh; Bleed |
| IHC | Day 15 - A. acervulina infection; |
| Day 7 - Boosting | Weigh; Bleed |
| Day 8 - A. acervulina infection | Day 21 - Place shedding tray, |
| Day 10-11 - Bleed | Day 22 - Conduct lesion scoring; |
| Day 12 - Weigh | Bleed; Weigh |
| Day 14 - Bleed; Place shedding | Day 24 - Collect shedding |
| tray, Harvest intestine for lesion | Day 29 - Bleed; Weigh |
| scoring; Conduct lesion score | |
| Day 17 - Weigh; Collect shedding | |
| Day 22 - Bleed | |

Targeted Delivery of the Vaccine:

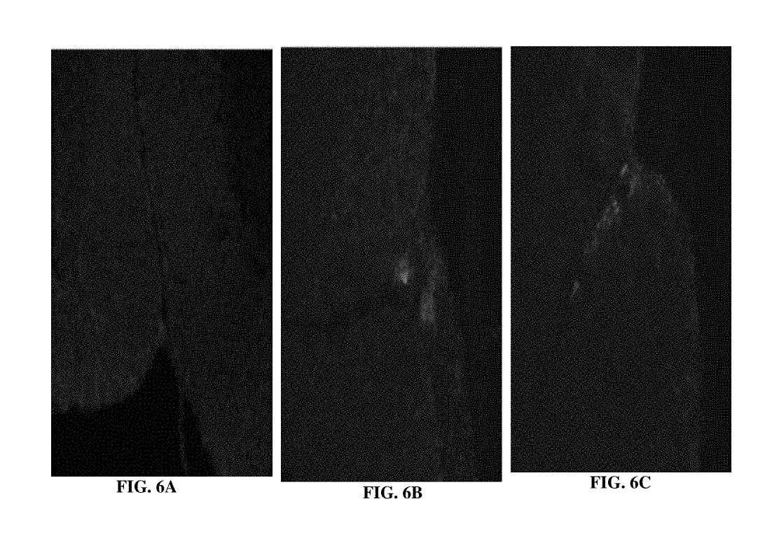

Frozen sections of intestinal tissues of chickens vaccinated with orally delivered 3-1e vaccine were stained with control serum, 3-1e pAb or E. coli LPS mAb. Goat anti-rabbit Alexa 488 (green) was used as secondary antibody. DAPI was used as a nuclear counterstain. The bacterial carrier has been shown effective to deliver the 3-1e vaccine to the digestive track (i.e., crop) of chicks 20 hours post inoculation (FIGS. 6A-6C). No positive signal was found in other intestinal areas (i.e., duodenum, jejunum, ileum and cecum) after 20 and 48 hours vaccination.

Vaccine Serological Response:

Blood samples were collected from the wing, or via cardiac puncture immediately following euthanasia. Sera was separated by centrifuging at 1,000 rpm for 20 min at 4° C. and stored at −20° C. until further use. Briefly, microtiter plates were coated with recombinant 3-1e protein at a concentration of 0.5 μg/well and incubated overnight at 4° C. (Schedule 1, N=3, Day 22, 14 DPI; Schedule 2, N=3, Day 22, 7 DPI).

The plates were washed with PBS-0.05% Tween, and blocked with PBS-1% BSA. 100 μL of serum (diluted 1:2-10 with PBS-T) were added to the wells and incubated for two hours. The plates were washed and 100 μL/well of peroxidase-conjugated rabbit anti-chicken IgY antibodies were added and incubated for 30 minutes, followed by color development with substrate. Optical density (OD) was determined at 450 nm with a microplate reader (Bio-Rad, Richmond, Calif.).

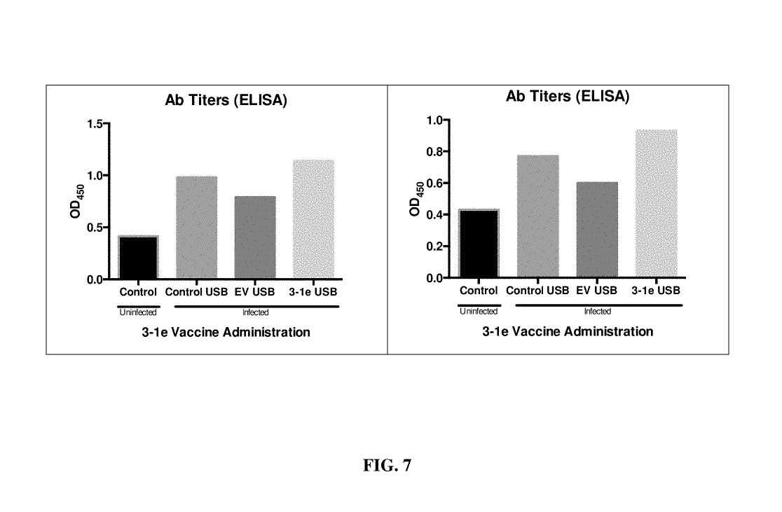

Chicks infected with E. acervulina and vaccinated with the orally delivered 3-1e vaccine showed highly increased 3-1e antibody levels compared to control uninfected and infected groups as well as control and EV infected groups (FIG. 7)

Gut Lesion Scoring:

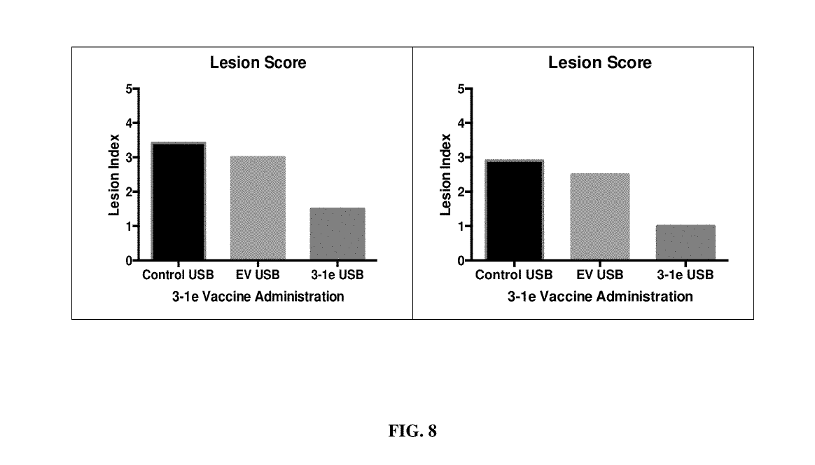

In both trials, three birds per group were euthanatized and approximately 20 cm intestinal segments (duodenum) extending 10 cm anterior and posterior to duodenal loop were obtained. Intestinal sections were scored for Eimeria lesions on a scale of 0 (none) to 4 (high) blindly by three independent observers, as a scoring metric to define the Eimeria-induced pathology upon the gut. The results demonstrate a nearly 2-fold reduction in the lesion scores in 3-1e vaccinates relative to the controls (Schedule 1, n=3; Schedule 2, n=3) (FIG. 8).

Oocyst Count:

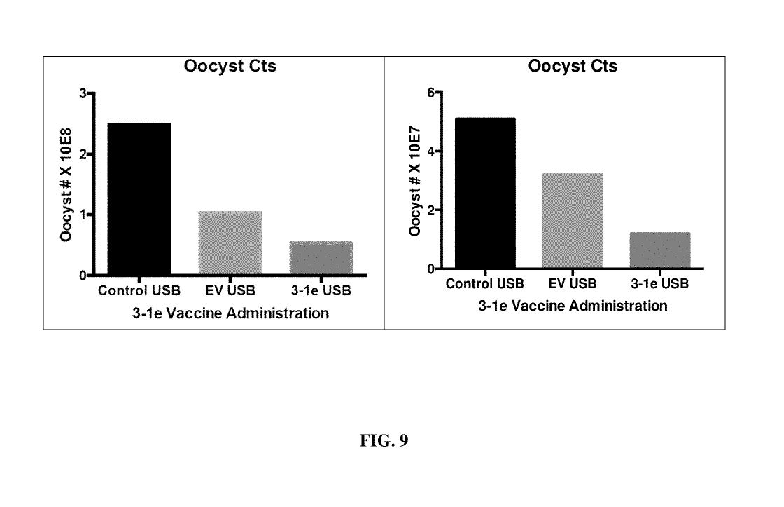

Oocysts were counted microscopically using a McMaster counting chamber using a sucrose flotation method, which has been established in the laboratory of Lillehoj. The total number of oocysts shed per chicken were calculated using the formula: total oocysts/bird=(oocyst count×dilution factor×fecal sample volume/counting chamber volume)/number of birds per cage. In the first trial, the orally delivered 3-1e vaccine resulted in nearly a log reduction of oocysts. In the second trial, the results indicated a nearly 4-fold reduction in oocyst shedding in the 3-1e vaccinate subjects (Schedule 1, N=6, Day 6-9 DPI; Schedule 2, N=8, Day 6-9 DPI) (FIG. 9).

Weight Gain:

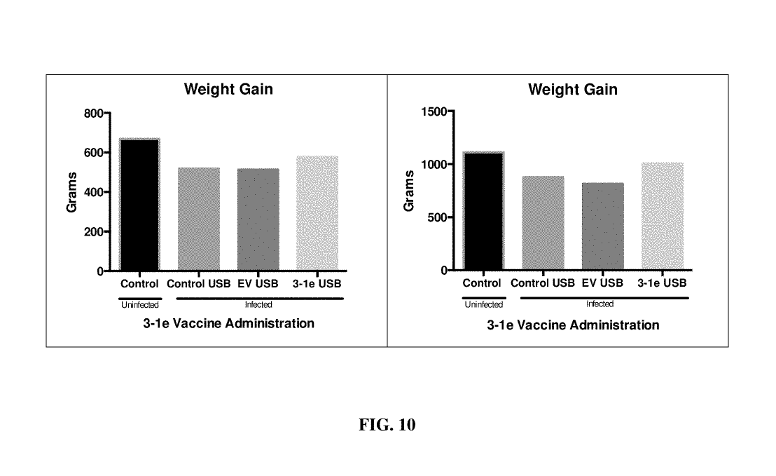

Compared to the uninfected controls, results of the 3-1e vaccinates demonstrate a slight increase in weight gain during the course of the two independent studies (Schedule 1, Gain Day 17-D 7; Schedule 2, Gain Day 29-D 15 (FIG. 10).

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A vaccine comprising a transformed host cell expressing a recombinant protein comprising SEQ ID NO: 2, or a protein having at least 95% identity to SEQ ID NO: 2, on its cell surface, wherein the recombinant protein is encoded by a nucleic acid used to transform the host cell, and a pharmacological carrier.

2. The vaccine of claim 1, wherein the host cell is an Escherichia coli cell.

3. The vaccine of claim 1, further comprising a probiotic organism of the genus Lactobacillus.

4. The vaccine of claim 3, wherein the probiotic organism is L. acidophilus, L. brevis, L. casei, L. crispatus, L. fermentum, L. gasseri, L. plantarum, L. reuteri, L. rhamnzosus, or L. salivarius.

5. The vaccine of claim 1, wherein the vaccine is a killed whole-cell vaccine.

6. The vaccine of claim 1, wherein the vaccine is a live whole-cell vaccine.

7. The vaccine of claim 1, wherein the pharmacological carrier is a hydrocolloid polymer, a plasticizing sugar, or a combination thereof.

8. The vaccine of claim 7, wherein the pharmacological carrier is sodium alginate.

9. The vaccine of claim 7, wherein the plasticizing sugar is sucrose or trehalose.

10. A vaccine comprising a transformed host cell expressing a recombinant protein comprising SEQ ID NO: 11, or a protein having at least 95% identity to SEQ ID NO: 11, wherein the recombinant protein is encoded by a nucleic acid used to transform the host cell, on its cell surface and a pharmacological carrier.

11. The vaccine of claim 10, wherein the host cell is an Escherichia coli cell.

12. The vaccine of claim 10, further comprising a probiotic organism of the genus Lactobacillus.

13. The vaccine of claim 10, wherein the vaccine is a killed whole-cell vaccine.

14. The vaccine of claim 10, wherein the vaccine is a live whole-cell vaccine.

15. The vaccine of claim 10, wherein the pharmacological carrier is a hydrocolloid polymer, a plasticizing sugar, or a combination thereof.

16. The vaccine of claim 15, wherein the pharmacological carrier is sodium alginate.

17. The vaccine of claim 15, wherein the plasticizing sugar is sucrose or trehalose.

18. A method of protecting a recipient against an Eimeria species, comprising: administering to a recipient the vaccine of claim 1 or claim 10 in an amount effective to induce an immune response against the exogenous protein presented by the vaccine.

19. The method of claim 18, wherein the recipient is poultry.

20. The method of claim 18, further comprising the step of administering to the recipient a probiotic organism of the genus Lactobacillus.

21. The method of claim 18, wherein the vaccine is administered to the recipient as a live whole-cell formulation at a dose of 5×10³ to 5×10⁹ CFU.

22. The method of claim 18, wherein the vaccine is administered to the recipient as a killed whole-cell formulation at a dose of 5×10³ to 5×10⁹ cells.
